(19) United States
(12) Patent Application Publication
(10) Pub. No.: US 2005/0059684 A1
(43) Pub. Date: Mar. 17, 2005

(54) Method for reducing residual alcohols in crystalline valacyclovir hydrochloride

(76) Inventors: Ben-Zion Dolitzky, Petach-Tiqva (IL); Igor Lifshitz, Petach Tiqva (IL)

Correspondence Address: KENYON & KENYON, ONE BROADWAY, NEW YORK, NY, 10004, US

(21) Appl. No.: 10/880,862

(22) Filed: Jun. 30, 2004

Related U.S. Application Data
(63) Continuation-in-part of application No. 10/688,538, filed on Oct. 16, 2003.
(60) Provisional application No. 60/419,270, filed on Oct. 16, 2002.
(60) Provisional application No. 60/427,320, filed on Nov. 18, 2002.

(51) Int. Cl. A61K 31/522; C07D 473/12

(52) U.S. Cl. 514/263.38; 544/276

(57) ABSTRACT

Provided is valacyclovir hydrochloride stable against formation of N′-formylvalacyclovir upon storage at elevated humidity and pharmaceutical compositions including such valacyclovir hydrochloride.

### CROSS-REFERENCE TO RELATED APPLICATIONS

This application is a continuation-in-part of U.S. patent application Ser. No. 10/688,538 filed Oct. 16, 2003, which claims the benefit of U.S. Provisional Patent Application No. 60/419,270, filed Oct. 16, 2002 and U.S. Provisional Application Ser. No. 60/427,320, filed Nov. 18, 2002, all of which are incorporated herein by reference.

### BACKGROUND OF THE INVENTION

Valacyclovir is an L-valyl ester prodrug of acyclovir. Acyclovir is an acyclic analog of a natural nucleoside which has been found to have high anti-viral activity. Acyclovir is widely used in the treatment and prophylaxis of viral infections in humans, particularly infections caused by the herpes group of viruses. See Goodman and Gilman's, The Pharmacological Basis of Therapeutics 1193-1198 (9th ed. 1996).

Acyclovir is an acyclic guanine nucleoside analog that lacks a 3′-hydroxyl on the side chain. Acyclovir has the chemical name 6H-Purin-6-one, 2-amino-1,9-dihydro-9-[(2-hydroxyethoxy)methyl]. (CAS Registry No. 59277-89-3.) Acyclovir as the sodium salt is currently marketed as ZOVIRAX®. The chemical structure of acyclovir is shown as Formula I.

Valacyclovir has the chemical name 1-valine, 2-[(2-amino-1,6-dihydro-6-oxo-9H-purin-9-yl)methoxy]ethyl ester. (CAS Registry No. 124832-26-4.) Valacyclovir is currently marketed as VALTREX®. The chemical structure of valacyclovir is shown as Structure I.

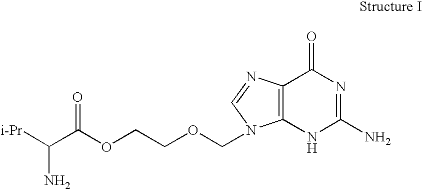

For oral administration, it is advantageous to administer valacyclovir rather than acyclovir because acyclovir is poorly absorbed from the gastrointestinal tract after oral administration in both animals and humans. In contrast, valacyclovir is rapidly absorbed from the gastrointestinal tract after oral administration. Moreover, valacyclovir is converted rapidly and virtually completely to acyclovir after oral administration in healthy adults. The conversion of valacyclovir is thought to result from first-pass intestinal and hepatic metabolism through enzymatic hydrolysis.

Work-up, isolation, and purification procedures for valacyclovir hydrochloride can and frequently do use solvents that are or that contain alcohols such as methanol, ethanol or iso-propanol. U.S. Pat. No. 4,957,924 discloses one such crystallization procedure that uses ethanol. In such cases, when alcohols are used in work-up or other procedures, the valacyclovir hydrochloride can contain 5000 ppm or more of excess residual process alcohol. The presence of unnecessary foreign substances, for example excess residual process alcohols, in any active pharmaceutical ingredient (API) is undesirable. These excess residual process alcohols are not necessary to the efficacy of the API valacyclovir hydrochloride. The solvents may be toxic and can produce undesirable effects in the patient receiving valacyclovir hydrochloride. Since there is no therapeutic benefit from residual process solvents, all residual solvents should be removed to the extent possible to meet quality-based requirements.

Indeed, health regulatory agencies in many countries have established limits for foreign substances in active pharmaceutical ingredients and may require manufacturers to adapt manufacturing procedures to reduce or eliminate them. For example, the United States Food and Drug Administration has promulgated guidelines (Q3C) that apply to residual solvents in drug substances and drug products.

The International Conference on Harmonization of Technical Requirements for Registration of Pharmaceuticals for Human Use has also promulgated Draft Guidelines (Q3C) for Residual Solvents in Pharmaceuticals. See Step 4 Draft, Jul. 16, 1997, Dr. Shigeo Kojima, rapporteur (hereafter ICH Guidelines). The draft proposes three classes of solvents and several options for quantifying the permissible level of them. Class 3 solvents should be limited (Option 1) to 5000 ppm, provided that the total daily dosage would be less than 50 mg (concentration in tablet should not exceed 5000 ppm). Ethanol and the propanols are among the class 3 solvents that should be limited by good manufacturing procedures (GMP).

Also, residual process solvents can be associated with deleterous effects on the “shelf-life” of active pharmaceutical ingredients (like valacyclovir and its salts) due to build-up of undesired impurities. In the case of valacyclovir and its salts, N′-formylvalacyclovir (i.e. 9-(2-(N′-formyl)valeryloxy)ethoxymethyl-2-amino-1,6-dihydro-6-oxo-9H-purine) is an impurity that may form more readily upon storage of valacyclovir having residual solvents.

Although some residual process solvent in an API or drug product may be an unavoidable consequence of the manufacturing process, the level of residual process solvent should be reduced to a minimum. Clearly, methods for reducing excess process solvents like alcohols in valacyclovir hydrochloride to a level less than 5000 ppm are needed.

### SUMMARY OF THE INVENTION

In one aspect, the present invention relates to a method of reducing excess residual process solvent (>5000 ppm), especially excess residual process alcohol, in valacyclovir hydrochloride having excess residual process solvent, including the step of statically or dynamically contacting the valacyclovir hydrochloride having excess residual process alcohol with a humid gas, especially humid air.

In another aspect, the present invention relates to a method of reducing excess residual process alcohol, especially excess residual process isopropanol, in valacyclovir hydrochloride having excess residual process alcohol including the step of dynamically contacting such valacyclovir hydrochloride with humid air, especially humid air of ≧50% relative humidity, more especially≧75% relative humidity, in a fluiduzed bed apparatus.

In another aspect, the present invention relates to valacyclovir hydrochloride that is substantially free of N′-formylvalacyclovir and especially to valacyclovir have less than 0.1% and most especially about 0.03% or less of N′-formylvalacyclovir.

In a further aspect, the present invention relates to valacyclovir hydrochloride that does not have excess residual process alcohol and that is stable against formation of (i.e. resists formation of) impurities, especially N′-formylvalacyclovir when stirred at elevated humidity, especially at 25° C. and 60% relative humidity (RH) and most especially when stored at 40° C. and 75% relative humidity.

In a further aspect, the present invention relates to valacyclovir hydrochloride that does not have excess residual process alcohol and that is sufficiently stable against formation of (i.e. resistant to formation of) impurities, especially N′-formylvalacyclovir hydrochloride, such that the amount of N′-formylvalacyclovir hydrochloride in the valacyclovir remains essentially unchanged when the valacyclovir hydrochloride is stored at elevated humidity, especially at 25° C. and 60% relative humidity (RH); and increases by no more than about 166% (based on the initial amount) when the valacyclovir is stored under the elevated humidity conditions of 40° C. and 75% relative humidity (RH).

In yet another aspect, the present invention related to a method of making valacyclovir hydrochloride that is stable against formation of N′-formylvalacyclovir hydrochloride when stored at elevated humidity, especially 25° C. and 60% RH, more especially at 40° C. and 75% RH, which method includes the step of statically or dynamically contacting valacyclovir hydrochloride, especially valacyclovir hydrochloride having excess residual process solvent (especially alcohol process solvent), with a humid gas, especially humid air, of at least 15% RH, especially at least 50% RH, most especially at least 75% RH.

### DETAILED DESCRIPTION OF THE INVENTION

One skilled in the art of the synthesis of organic compounds understands that solvents, e.g. alcohols, are often used in synthesis procedures and that traces, sometimes substantial traces, of these solvents can remain in the compound synthesized. The remaining solvents, which can be referred to as residual process solvents, can be difficult to remove. The present inventors have discovered that, in the case of valacyclovir and its hydrochloride salt, these residual process solvents, especially residual process alcohols, may be associated with chemical instability in the product. Specifically, the present invenotrs have found that residual process alcohols in valacyclovir hydrochloride can be correlated with an increased propensity to formation of the impurity 9-(2-(N′-formyl)valeryloxy)ethoxymethyl-2-amino-1,6-dihydro-6-oxo-9H-purine (N′-formylvalacyclovir, Structure II), or its hydrochloride salt, upon storage, especially at elevated humidity.

When used herein in connection with the absolute amount of an impurity, percent refers to area percent of the corresponding impurity peak on the chromatogram obtained by high pressure liquid chromatography.

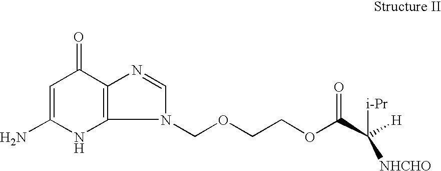

Residual process solvents in pharmaceutical compounds (and ultimately any pharmaceutical compositions prepared therefrom) serve no therapeutic purpose and can be harmful to the patient. As discussed above, governmental regulatory agencies and international advisory organizations have promulgated regulations and guidelines for residual (process) solvents in pharmaceutical compounds. Moreover, as discussed above, residual process solvents in an API may be correlated with an increased propensity of the API to form impurities on storage, especially storage at elevated humidity.

Excess residual process solvent in valacyclovir hydrochloride is defined in relation to the concentration limits set for class 3 solvents, of which iso-propanol is one example, by the ICH Guidelines. Accordingly, excess process alcohol in valacyclovir hydrochloride refers to process alcohol, especially ethanol and iso-propanol, in excess of 5000 ppm on a weight basis. Valacylcovir hydrochloride having excess residual process alcohol refers to valacyclovir hydrochloride having 5000 ppm or more, on a weight basis, residual process alcohol. Valacyclovir hydrochloride having excess residual process alcohol is a preferred starting material for use in the practice of the method of the present invention.

Alcohols can be used as solvents in the synthesis, work-up, and purification of valacyclovir hydrochloride. The present invention provides a method for reducing the excess residual process alcohol content of crystalline valacyclovir hydrochloride having excess residual process alcohols, for example ethanol, n-propanol, or iso-propanol, remaining from, for example, work-up, isolation, or other treatment procedures, for example recrystallization.

Valacyclovir hydrochloride is considered to have excess residual process alcohol if the residual process alcohol is ≧5000 ppm on a weight basis. The valacyclovir hydrochloride having residual process alcohol, especially excess residual process alcohol (residual process alcohol of 5000 ppm or more) can be from any source. Typically, the valacyclovir hydrochloride will be obtained from a process in which an alcohol or alcohol-containing solvent is used, for example from a crystallization procedure in which an alcohol is used. But valacyclovir hydrochloride having excess residual process alcohol can be obtained directly from a synthesis process in which an alcohol is used. In such cases, the excess process alcohol can be more than 5000 ppm.

The present method includes the step of contacting (exposing) particles (e.g. individual crystals) of valacyclovir hydrochloride having excess residual process alcohol with a humid gas, preferably at ambient atmospheric pressure (about 750 to about 765 mm Hg).

Any gas that does not induce or accelerate chemical degradation of valacyclovir hydrochloride during the contacting process can be used. Air is the preferred gas. Humid gas has a relative humidity (RH) of at least about 15%, preferably at least about 50%, more preferably at least about 75%. Relative humidity refers to the ratio (times 100) of the actual vapor pressure of water in a gas to the saturation vapor pressure of water in the gas at a particular temperature and pressure.

The contacting is conducted at ambient pressure and a temperature of about 10° C. to about 60° C. The contacting can be static or it can be dynamic.

In static contacting, particles of valacyclovir hydrochloride are at rest. That is, they are not mechanically or otherwise agitated or stirred. Static contacting can be carried out, for example, by contacting particles of valacyclovir hydrochloride having excess residual process alcohol supported on a tray, preferably in a thin layer, with humid gas in a suitable enclosure such as, for example, a constant humidity chamber.

In dynamic contacting, particles of valacyclovir hydrochloride are in motion induced by mechanical or other agitation whilst being contacted with humid gas. Mechanical agitation can be provided by, for example, a ribbon-type blender through which humid gas is passed.

In a preferred embodiment, the valacyclovir hydrochloride having excess residual process alcohol is contacted with humid gas in a fluidized bed apparatus wherein the valacyclovir hydrochloride is fluidized with the humid gas. Fluidized bed apparatus is well-known in the art. One example of suitable fluidized bed apparatus is a Retsch model TG-100.

Valacyclovir hydrochloride having residual process alcohol, especially excess residual process alcohol, is contacted with humid air for a contacting time sufficient to reduce the residual process alcohol to less that 5000 ppm, preferably to about 1000 ppm or less. The skilled artisan will know to optimize the contacting time by routine experimentation, taking into consideration factors such as the amount of residual process alcohol or residual process alcohol initially present, the size of the particles of valacyclovir hydrochloride, and the humidity of the humid gas. The higher the initial amount of excess residual process alcohol, the larger the particles of valacyclovir hydrochloride, and the lower the humidity of the humid gas, the longer will be, in general, the contacting time. The lower the initial amount of excess residual process alcohol and the higher the humidity of the humid gas, the shorter, in general, will be the contacting time.

By the method of the present invention, the excess residual process alcohol in valacyclovir hydrochloride is reduced to <5000 ppm, preferably to about 1000 ppm or less. Alcohol in valacyclovir hydrochloride can be measured by any means known in the art, for example by gas chromatography (GC).

Valacyclovir hydrochloride treated by the method of the present invention is stable against formation or build-up of (i.e. is relatively resistant to formation of) N′-formylvalacyclovir hydrochloride (hereafter simply N′-formylvalacyclovir) upon storage at elevated humidity (>50% RH), and especially when stored at 25° C. and 60% RH or more especially, 40° C. and 75% RH for about 3 months or longer. Valacyclovir hydrochloride is stable against formation of N′-formyl valacyclovir upon storage at elevated humidity if the amount of N′-formylvalacyclovir (as measured by HPLC) remains essentially unchanged upon storage for about 3 months, or increases to no more than 175%, preferably no more than 166%, of its initial value, remaining in any event below about 0.1 area %, preferably below about 0.07 area %. Valacyclovir hydrochloride is considered to be substantially free of N′-formylvalacyclovir if it has less than about 0.1 area % N′-formylvalacyclovir.

Impurities in valacyclovir, especially N′-formylvalacyclovir, can be measure by high pressure liquid chromatography. The chromatographic method utilizes a suitable chromatography column such as the reverse phase column Inertsil ODS-3V 5 μm 150×4.6 mm (GL Sciences, Cat. No. 5020-01731). The eluent is preferably acetonitrile (27%) and water (73%) containing 0.05% phosphoric acid (0.5 g 85% H₃PO₄ in 1 L water). A flow rate of about 1.0 mL/min is suitable for the separation. The column temperature can be at ambient temperature, preferably about 25° C. The detector can be a UV-spectrophotometer operating in the range of 200-600 nm, preferably about 210 nm. The injection (sample) volume is about 50 μL. The diluent used to load the sample onto the column can be, for example, the eluent.

Valacyclovir hydrochloride may be formulated into a variety of pharmaceutical compositions and dosage forms that are useful in treating patients afflicted with viral infections, particularly infections caused by the herpes group of viruses.

In one embodiment, the present invention relates to pharmaceutical compositions including valacyclovir hydrochloride in at least one of forms I, II, IV, V, VI or VII. In addition to the active ingredient(s), valacyclovir hydrochloride pharmaceutical compositions of the present invention may contain one or more excipients. Excipients are added to the composition for a variety of purposes.

Diluents increase the bulk of a solid pharmaceutical composition and may make a pharmaceutical dosage form containing the composition easier for the patient and caregiver to handle. Diluents for solid compositions include, for example, microcrystalline cellulose (e.g. AVICEL®, microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form like a tablet may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) and starch.

Glidants can be added to improve the flow properties of non-compacted solid compositions and improve the accuracy of dosing. Excipients that may function as glidants include colloidal silicon dixoide, magnesium trisilicate, powdered cellulose, starch, talc and tribasic calcium phosphate.

When a dosage form such as a tablet is made by compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl famarate, stearic acid, talc and zinc stearate.

Flavoring agents and flavor enhancers make the dosage form more palatable to the patient. Conunon flavoring agents and flavor enhancers for pharmaceutical products that may be included in the composition of the present invention include maltol, vanillin, ethyl vanillin, menthol, citric acid, fumaric acid ethyl maltol, and tartaric acid.

Compositions may also be colored using any pharmaceutically acceptable colorant to improve their appearance and/or facilitate patient identification of the product and unit dosage level.

Selection of excipients and the amounts to use may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable route in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges as well as liquid syrups, suspensions and elixirs. An especially preferred dosage form of the present invention is a tablet.

Tablets, capsules, lozenges and other unit dosage forms preferably contain modafinil in a dosage level of from about 50 to about 300 mg, more preferably from about 100 mg to about 200 mg.

The currently marketed form of valacyclovir (VALTREX®) contains valacyclovir hydrochloride equivalent to 500 mg valacyclovir and the inactive ingredients carnauba wax, colloidal silicon dioxide crospovidone, FD&C Blue No. 2 Lake, hydroxypropyl methylcellulose, magnesium stearate, microcrystalline cellulose polyethylene glycol, polysorbate 80, povidone and titanium dioxide.

The present invention can be illustrated with the following nonlimiting examples.

### EXAMPLE 1

Valacyclovir hydrochloride (about 10 g), crystallized from isopropanol/water and having about 6000 ppm excess residual process solvent, were dried in a fluidized bed drier at about 40° C. for about 4 hours in a stream of humid air (ca. 80% RH). After drying, the material so treated contained less than about 300 ppm residual process solvent and about 9% by weight water.

### EXAMPLE 2

A sample of valacyclovir hydrochloride (sample X) having circa 1700 ppm residual solvent isopropanol was divided into two portions. One portion was treated by the method of the present invention and, after treatment, had less than 500 ppm residual isopropanol (sample Y). Sample Y and the non-treated portion of sample X were each divided into two roughly equal portions. One portion of each samplewas stored at 25° C. and 60% RH for three months, the other portions of each were stored at 40° C. and 75% RH for three months. The amount of N′-formylvalacyclovir in each of the four portions after storage was determined by HPLC. The results are given below.

| | | N′-formylvalacyclovir level (area %) | | |
|---|---|---|---|---|
| | | | T = 3 month, | T = 3 month, |
| | Residual | | 25° C., 60% | 40° C., 75% |
| Batch No. | solvent (ppm) | T = 0 | humidity | humidity |
| X | 1671 | 0.05 | 0.08 | 0.15 |
| Y | 497 | 0.03 | 0.03 | 0.05 |

1. Valacyclovir hydrochloride stable against formation of N′-formylvalacyclovir upon storage at elevated humidity.

2. The valacyclovir hydrochloride of claim 1 wherein the storage at elevated humidity is at about 25° C. and 60% RH.

3. The valacyclovir hydrochloride of claim 2 wherein the time of storage is about 3 months and the amount of N′-formylvalacyclovir in the valacyclovir is essentially unchanged during this storage time.

4. The valacyclovir hydrochloride of claim 1 wherein the storage at elevated humidity is at about 40° C. and about 75% RH.

5. The valacyclovir hydrochloride of claim 1 wherein the storage at elevated humidity is for a storage time of about 3 months and the amount of N′-formylvalacyclovir in the valacyclovir increases during the storage time to not more than about 166% of its initial value during this storage time.

6. The valacyclovir hydrochloride of claim 1 wherein the amount of N′-formylvalacyclovir after storage is about 0.1% or less.

7. The valacyclovir hydrochloride of claim 6 wherein the amount of N′-formylvalacyclovir after storage is about 0.07% or less.

8 A method of making valacyclovir hydrochloride stable against formation of N′-formylvalacyclovir upon storage at elevated humidity comprising the step of contacting valacyclovir hydrochloride having excess residual process alcohol with a humid gas at ambient pressure.

9. The method of claim 8 wherein the humid gas is humid air.

10. The method of claim 8 wherein the humid gas has a relative humidity of at least about 15%.

11. The method of claim 10 wherein the humid gas has a relative humidity of at least about 50%.

12. The method of claim 11 wherein the humid gas has a relative humidity of at least about 75%.

13. The method of claim 8 wherein the contacting is static.

14. The method of claim 8 wherein the contacting is dynamic.

15. The method of claim 8 wherein the contacting is in a fludized bed apparatus.

16. A pharmaceutical composition comprising valacyclovir hydrochloride stable against formation of N′-formylvalacyclovir upon storage at elevated humidity and at least one pharmaceutically acceptable excipient.

17. The pharmaceutical composition of claim 16 wherein the storage at elevated humidity is at 25° C. and 60% RH.

18. The pharmaceutical composition of claim 16 wherein the storage at elevated humidity is at about 45° C. and about 75% RH.

19. A pharmaceutical composition comprising at least one pharmaceutically acceptable excipient and valacyclovir hydrochloride stable against formation of N′-formylvalacyclovir upon storage for about 3 months at 40° C. and 75% RH, wherein, upon such storage, the amount of N′-formylvalacyclovir hydrochloride in the valacyclovir hydrochloride increases to no more than about 166% of its initial value.
